(19) United States
(12) Patent Application Publication
(10) Pub. No.: US 2005/0153424 A1
(43) Pub. Date: Jul. 14, 2005

(54) Fluid barrier with transparent areas for immersion lithography

(76) Inventor: Derek Coon, Redwood City, CA (US)

Correspondence Address: The Law Office of Steven G. Roeder, 5560 Chelsea Avenue, La Jolla, CA, 92037, US

(21) Appl. No.: 10/754,793

(22) Filed: Jan. 8, 2004

(51) Int. Cl. G03B 27/00; C12M 1/34

(52) U.S. Cl. 435/287.1; 355/403

(57) ABSTRACT

A fluid supply system (26) for controlling an environment in a gap (246) between an optical assembly (16) and a device (30) includes a fluid source (372) and a fluid barrier (256). The fluid source (372) directs an immersion fluid (248) into the gap (246). The fluid barrier (256) is positioned near the gap (246). Further, the fluid barrier (256) includes a transparent area (598) that is substantially transparent. With this design, a measurement system (22) can direct a light beam (270) through the fluid barrier (256) to monitor the position of the device (30).

### FIELD OF THE INVENTION

The present invention is directed to a fluid barrier for an immersion lithography system.

### BACKGROUND

Exposure apparatuses are commonly used to transfer images from a reticle onto a semiconductor wafer during semiconductor processing. A typical exposure apparatus includes an illumination source, a reticle stage assembly that positions a reticle, an optical assembly, a wafer stage assembly that positions a semiconductor wafer, and a measurement system that precisely monitors the position of the reticle and the wafer.

Immersion lithography systems require that a layer of immersion fluid completely fill a gap between the optical assembly and the wafer. In one design, the immersion fluid is retained in the gap with a fluid barrier that encircles the gap.

Unfortunately, the fluid barrier limits the effectiveness of some of the other components of the exposure apparatus, and complicates the design of the other components. For example, the fluid barrier may severely limit the effectiveness of the measurement system to measure the position of the wafer. This reduces the accuracy of positioning of the wafer relative to the reticle and degrades the accuracy of the exposure apparatus.

### SUMMARY

The present invention is directed to a fluid immersion system for controlling an environment in a gap between an optical assembly and a device. In one embodiment, the fluid immersion system includes a fluid source and a fluid barrier. The fluid source can direct an immersion fluid into the gap. The fluid barrier is positioned near the gap. In one embodiment, the fluid barrier includes a transparent area that is made from a substantially transparent material. In one embodiment, the substantially transparent material has a coefficient of extinction that is relatively small and close to zero. In alternative embodiments, the substantially transparent material has a coefficient of extinction of less than approximately 0.2, 0.1, 0.08, 0.06, 0.04, 0.02 or 0.01.

In one embodiment, the transparent material has an index of refraction that is not equal to the index of refraction of the immersion fluid. In another embodiment, the transparent material has an index of refraction that is approximately equal to an index of refraction of the immersion fluid. This embodiment can be useful if the fluid barrier is not substantially perpendicular to a beam from a measurement system. In alternative embodiments, the transparent material has an index of refraction that is within at least approximately 0.1, 0.2, 0.3, 0.4, 0.5, or 0.6 of an index of refraction of the immersion fluid. In yet another embodiment, the transparent material has an index of refraction that is within approximately 50 percent of an index of refraction of the immersion fluid. In alternative embodiments, the transparent material has an index of refraction that is within approximately 1, 2, 3, 4, 5, or 10 percent of an index of refraction of the immersion fluid.

The present invention is also directed to an exposure apparatus, a wafer, a device, a method for controlling an environment in a gap, a method for making an exposure apparatus, a method for making a device and a method for manufacturing a wafer.

### BRIEF DESCRIPTION OF THE DRAWINGS

FIG. 1 is a side illustration of an exposure apparatus having features of the present invention;

FIG. 2A is a side illustration of a portion of the exposure apparatus of FIG. 1;

FIG. 2B is a partial cut-away perspective illustration of a portion of the exposure apparatus of FIG. 1;

FIG. 3 is a top plan view of a fluid immersion system having features of the present invention;

FIG. 4 is a perspective view of a portion of the fluid immersion system of FIG. 3;

FIG. 5 is a cut-away view taken on line 5-5 of FIG. 3;

FIG. 6A is a flow chart that outlines a process for manufacturing a device in accordance with the present invention; and

FIG. 6B is a flow chart that outlines device processing in more detail.

### DESCRIPTION

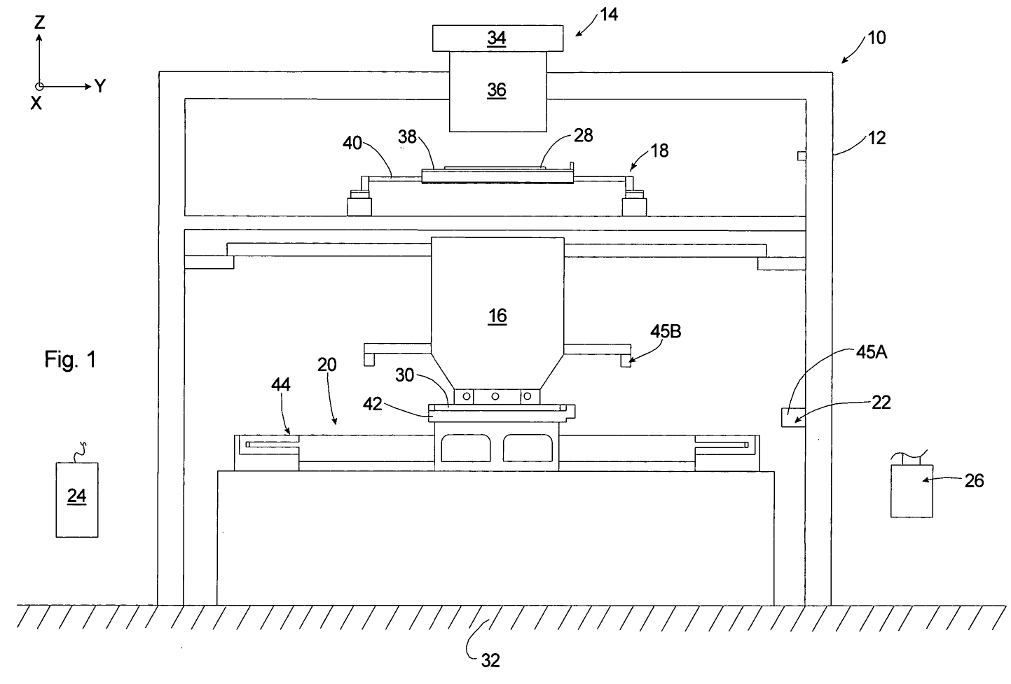

FIG. 1 is a schematic illustration of a precision assembly, namely an exposure apparatus 10 having features of the present invention. The exposure apparatus 10 includes an apparatus frame 12, an illumination system 14 (irradiation apparatus), an optical assembly 16, a reticle stage assembly 18, a wafer stage assembly 20, a measurement system 22, a control system 24, and a fluid supply system 26.

A number of Figures include an orientation system that illustrates an X axis, a Y axis that is orthogonal to the X axis, and a Z axis that is orthogonal to the X and Y axes. It should be noted that these axes can also be referred to as the first, second and third axes.

The exposure apparatus 10 is particularly useful as a lithographic device that transfers a pattern (not shown) of an integrated circuit from a reticle 28 onto a semiconductor wafer 30. The exposure apparatus 10 mounts to a mounting base 32, e.g., the ground, a base, or floor or some other supporting structure.

There are a number of different types of lithographic devices. For example, the exposure apparatus 10 can be used as a scanning type photolithography system that exposes the pattern from the reticle 28 onto the wafer 30 with the reticle 28 and the wafer 30 moving synchronously. In a scanning type lithographic device, the reticle 28 is moved perpendicularly to an optical axis of the optical assembly 16 by the reticle stage assembly 18 and the wafer 30 is moved perpendicularly to the optical axis of the optical assembly 16 by the wafer stage assembly 20. Scanning of the reticle 28 and the wafer 30 occurs while the reticle 28 and the wafer 30 are moving synchronously.

Alternatively, the exposure apparatus 10 can be a step-and-repeat type photolithography system that exposes the reticle 28 while the reticle 28 and the wafer 30 are stationary. In the step and repeat process, the wafer 30 is in a constant position relative to the reticle 28 and the optical assembly 16 during the exposure of an individual field. Subsequently, between consecutive exposure steps, the wafer 30 is consecutively moved with the wafer stage assembly 20 perpendicularly to the optical axis of the optical assembly 16 so that the next field of the wafer 30 is brought into position relative to the optical assembly 16 and the reticle 28 for exposure. Following this process, the images on the reticle 28 are sequentially exposed onto the fields of the wafer 30, and then the next field of the wafer 30 is brought into position relative to the optical assembly 16 and the reticle 28.

However, the use of the exposure apparatus 10 provided herein is not limited to a photolithography system for semiconductor manufacturing. The exposure apparatus 10, for example, can be used as an LCD photolithography system that exposes a liquid crystal display device pattern onto a rectangular glass plate or a photolithography system for manufacturing a thin film magnetic head.

The apparatus frame 12 supports the components of the exposure apparatus 10. The apparatus frame 12 illustrated in FIG. 1 supports the reticle stage assembly 18, the wafer stage assembly 20, the optical assembly 16 and the illumination system 14 above the mounting base 32.

The illumination system 14 includes an illumination source 34 and an illumination optical assembly 36. The illumination source 34 emits a beam (irradiation) of light energy. The illumination optical assembly 36 guides the beam of light energy from the illumination source 34 to the optical assembly 16. The beam illuminates selectively different portions of the reticle 28 and exposes the wafer 30. In FIG. 1, the illumination source 34 is illustrated as being supported above the reticle stage assembly 18. Typically, however, the illumination source 34 is secured to one of the sides of the apparatus frame 12 and the energy beam from the illumination source 34 is directed to above the reticle stage assembly 18 with the illumination optical assembly 36.

The optical assembly 16 projects and/or focuses the light passing through the reticle 28 to the wafer 30. Depending upon the design of the exposure apparatus 10, the optical assembly 16 can magnify or reduce the image illuminated on the reticle 28. The optical assembly 16 need not be limited to a reduction system. It could also be a 1× or magnification system.

Also, with an exposure device that employs vacuum ultra-violet radiation (VUV) of wavelength 200 nm or lower, use of the catadioptric type optical system can be considered. Examples of the catadioptric type of optical system include the disclosure Japan Patent Application Disclosure No. 8-171054 published in the Official Gazette for Laid-Open Patent Applications and its counterpart U.S. Pat. No. 5,668,672, as well as Japan Patent Application Disclosure No. 10-20195 and its counterpart U.S. Pat. No. 5,835,275. In these cases, the reflecting optical device can be a catadioptric optical system incorporating a beam splitter and concave mirror. Japan Patent Application Disclosure No. 8-334695 published in the Official Gazette for Laid-Open Patent Applications and its counterpart U.S. Pat. No. 5,689,377 as well as Japan Patent Application Disclosure No. 10-3039 and its counterpart U.S. patent application Ser. No. 873,605 (Application Date: Jun. 12, 1997) also use a reflecting-refracting type of optical system incorporating a concave mirror, etc., but without a beam splitter, and can also be employed with this invention. As far as is permitted, the disclosures in the above-mentioned U.S. patents, as well as the Japan patent applications published in the Official Gazette for Laid-Open Patent Applications are incorporated herein by reference.

The reticle stage assembly 18 holds and positions the reticle 28 relative to the optical assembly 16 and the wafer 30. In one embodiment, the reticle stage assembly 18 includes a reticle table 38 that retains the reticle 28 and a reticle stage mover assembly 40 that moves and positions the reticle table 38 and reticle 28.

Somewhat similarly, the wafer stage assembly 20 holds and positions the wafer 30 with respect to the projected image of the illuminated portions of the reticle 28. In one embodiment, the wafer stage assembly 20 includes a wafer table 42 that retains the wafer 30, and a wafer stage mover assembly 44 that moves and positions the wafer table 42 and wafer 28.

Each mover assembly 40, 44 can move the respective table 38, 42 with three degrees of freedom, less than three degrees of freedom, or more than three degrees of freedom. The reticle stage mover assembly 40 and the wafer stage mover assembly 44 can each include one or more movers, such as rotary motors, voice coil motors, linear motors utilizing a Lorentz force to generate drive force, electromagnetic movers, planar motors, or some other force movers.

In photolithography systems, when linear motors (see U.S. Pat. Nos. 5,623,853 or 5,528,118) are used in the wafer stage assembly or the reticle stage assembly, the linear motors can be either an air levitation type employing air bearings or a magnetic levitation type using Lorentz force or reactance force. Additionally, the stage could move along a guide, or it could be a guideless type stage that uses no guide. As far as is permitted, the disclosures in U.S. Pat. Nos. 5,623,853 and 5,528,118 are incorporated herein by reference.

Alternatively, one of the stages could be driven by a planar motor, which drives the stage by an electromagnetic force generated by a magnet unit having two-dimensionally arranged magnets and an armature coil unit having two-dimensionally arranged coils in facing positions. With this type of driving system, either the magnet unit or the armature coil unit is connected to the stage base and the other unit is mounted on the moving plane side of the stage.

Movement of the stages as described above generates reaction forces that can affect performance of the photolithography system. Reaction forces generated by the wafer (substrate) stage motion can be mechanically transferred to the floor (ground) by use of a frame member as described in U.S. Pat. No. 5,528,100 and published Japanese Patent Application Disclosure No. 8-136475. Additionally, reaction forces generated by the reticle (mask) stage motion can be mechanically transferred to the floor (ground) by use of a frame member as described in U.S. Pat. No. 5,874,820 and published Japanese Patent Application Disclosure No. 8-330224. As far as is permitted, the disclosures in U.S. Pat. Nos. 5,528,100 and 5,874,820 and Japanese Patent Application Disclosure No. 8-330224 are incorporated herein by reference.

The measurement system 22 monitors movement of the reticle 28 and the wafer 30 relative to the optical assembly 16 or some other reference. With this information, the control system 24 can control the reticle stage assembly 18 to precisely position the reticle 28 and the wafer stage assembly 20 to precisely position the wafer 30. The design of the measurement system 22 can vary. For example, the measurement system 22 can utilize multiple laser interferometers, encoders, and/or other measuring device. In the embodiment illustrated in FIG. 1, the measurement system 22 includes (i) an X/Y system 45A that measures the position of the wafer 30 along the X axis, along the Y axis and about the Z axis, and (ii) a Z system 45B that measures the position of the wafer 30 along the Z axis, about the X axis and about the Y axis. The measurement system 22 is further described below.

The control system 24 receives information from the measurement system 22 and controls the stage mover assemblies 18, 20 to precisely position the reticle 28 and the wafer 30. Additionally, the control system 24 can control the operation of the fluid supply system 26. The control system 24 can include one or more processors and circuits.

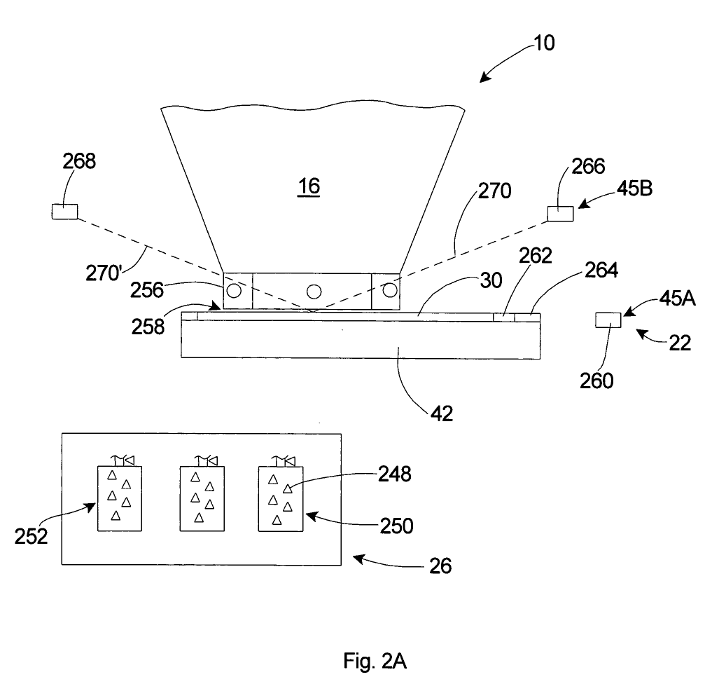

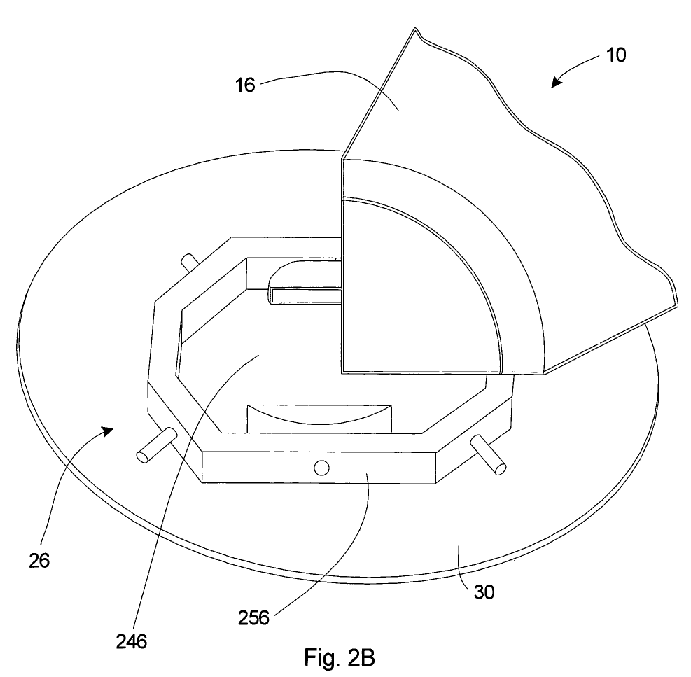

The fluid supply system 26 controls the environment in a gap 246 (illustrated in FIG. 2B) between the optical assembly 16 and the wafer 30. The gap 246 is also referred to herein as the exposure area. With this design, the fluid supply system 26 can control the environment in the area adjacent to the region of the wafer 30 that is being exposed and the area in which the beam of light energy travels between the optical assembly 16 and the wafer 30. For example, the fluid supply system 26 can direct an immersion fluid 248 (illustrated as triangles in FIG. 2A) into the gap 246 between the optical assembly 16 and the wafer 30. The fluid supply system 26 is described in more detail below.

A photolithography system (an exposure apparatus) according to the embodiments described herein can be built by assembling various subsystems, including each element listed in the appended claims, in such a manner that prescribed mechanical accuracy, electrical accuracy, and optical accuracy are maintained. In order to maintain the various accuracies, prior to and following assembly, every optical system is adjusted to achieve its optical accuracy. Similarly, every mechanical system and every electrical system are adjusted to achieve their respective mechanical and electrical accuracies. The process of assembling each subsystem into a photolithography system includes mechanical interfaces, electrical circuit wiring connections and air pressure plumbing connections between each subsystem. Needless to say, there is also a process where each subsystem is assembled prior to assembling a photolithography system from the various subsystems. Once a photolithography system is assembled using the various subsystems, a total adjustment is performed to make sure that accuracy is maintained in the complete photolithography system. Additionally, it is desirable to manufacture an exposure system in a clean room where the temperature and cleanliness are controlled.

FIG. 2A is a side view that illustrates a portion of the exposure apparatus 10 including the optical assembly 16, the wafer table 42, a portion of the fluid supply system 26, and a portion of the measurement system 22. In this embodiment, the fluid supply system 26 controls the environment in the gap 246 (illustrated in FIG. 2B) between the optical assembly 16 and the wafer 30. For example, the fluid supply system 26 can inject the immersion fluid 248 into the gap 246. The location of where the immersion fluid 248 is injected can vary. For example, the immersion fluid 248 can be introduced at multiple locations at or near the edge of the optical assembly 16. Alternatively, the immersion fluid 248 may be injected directly between the optical assembly 16 and the wafer 30.

In the embodiment illustrated in FIG. 2A, the fluid supply system 26 includes a fluid delivery system 250 and a fluid recovery system 252. In this embodiment, (i) the fluid delivery system 250 delivers the immersion fluid 248 into the gap 246, and (ii) the fluid recovery system 252 inhibits the immersion fluid 248 from flowing from the gap 246 and recovers the immersion fluid 248 released into the gap 246.

In this embodiment, the fluid recovery system 252 includes a fluid barrier 256. Further, in this embodiment, the fluid barrier 256 is secured to the bottom of the optical assembly 16 and the fluid barrier 256 is positioned above the wafer table 42 and the wafer 30. Additionally, in this embodiment, there is a movement gap 258 (greatly exaggerated in FIG. 2A) between the bottom of the fluid barrier 256 and the top of the wafer table 42 and the wafer 30 to allow for ease of movement of the wafer table 42 and the wafer 30 relative to the fluid barrier 256 and relatively small amount of leakage. For example, the movement gap 258 can be between approximately 0.5 and 2 millimeters.

FIG. 2A also illustrates the X/Y system 45A and the Z system 45B of the measurement system 22. In this embodiment, the X/Y system 45A includes (i) a first X interferometer (not shown), a second X interferometer (not shown), a Y interferometer 260, an X reflector 262 and a Y reflector 264. Each X interferometer generates a laser beam that is directed at the X reflector 262 and subsequently receives the beam that is reflected off of the X reflector 262. The Y interferometer 260 generates a laser beam that is directed at the Y reflector 264 and subsequently receives the beam that is reflected off of the Y reflector 264. In FIG. 2A, each reflector 262, 264 is a rectangular shaped, bar type mirror that is secured to the wafer table 42. In this embodiment, the X interferometers are used to measure the position of the wafer table 42 along the X axis and about the Z axis, and the Y interferometer 260 is used to measure the position of the wafer table 42 along the Y axis. Alternatively, for example, a single X interferometer and two Y interferometers can be utilized.

In FIG. 2A, the interferometers 260 are positioned away from the wafer table 42 and can be secured to the apparatus frame 12 (illustrated in FIG. 1) or the optical assembly 16 (illustrated in FIG. 1), as examples.

Additionally, FIG. 2A illustrates the Z system 45B. In this embodiment, the Z system 45B is an auto-focus system that includes a Z light source 266 and a Z detector 268. The Z light source 266 generates a light beam 270 (illustrated as dashed lines) that is directed through a portion of the fluid barrier 256 at the wafer 30. The light beam 270 is reflected off of the wafer 30 as reflected beam 270′ (illustrated as dashed lines) that also passes through a portion of the fluid barrier 256. The Z detector 268 receives the reflected beam 270′ and determines the position of the wafer 30 along the Z axis, and about the X and Y axes. As alternative examples, the light beam 270 can be at a wavelength of between approximately 530 and 800 nm.

In FIG. 2A, the Z light source 266 and the Z detector 268 are positioned away from the wafer table 42 and can be secured to the apparatus frame 12 (illustrated in FIG. 1) or the optical assembly 16 (illustrated in FIG. 1), as examples.

FIG. 2B is a partly cut-away perspective view of the optical assembly 16, a portion of the fluid supply system 26, and the wafer 30. Further, FIG. 2B illustrates that in this embodiment, the gap 246 between the optical assembly 16 and the wafer 30 is encircled by the fluid barrier 256. The desired environment created in the gap 246 by the fluid supply system 26 can vary accordingly to the wafer 30 and the design of the rest of the components of the exposure apparatus 10. For example, the desired controlled environment can be an inert gas such as Argon, Helium, or Nitrogen. Alternately, for example, the controlled environment can be water or some other fluid.

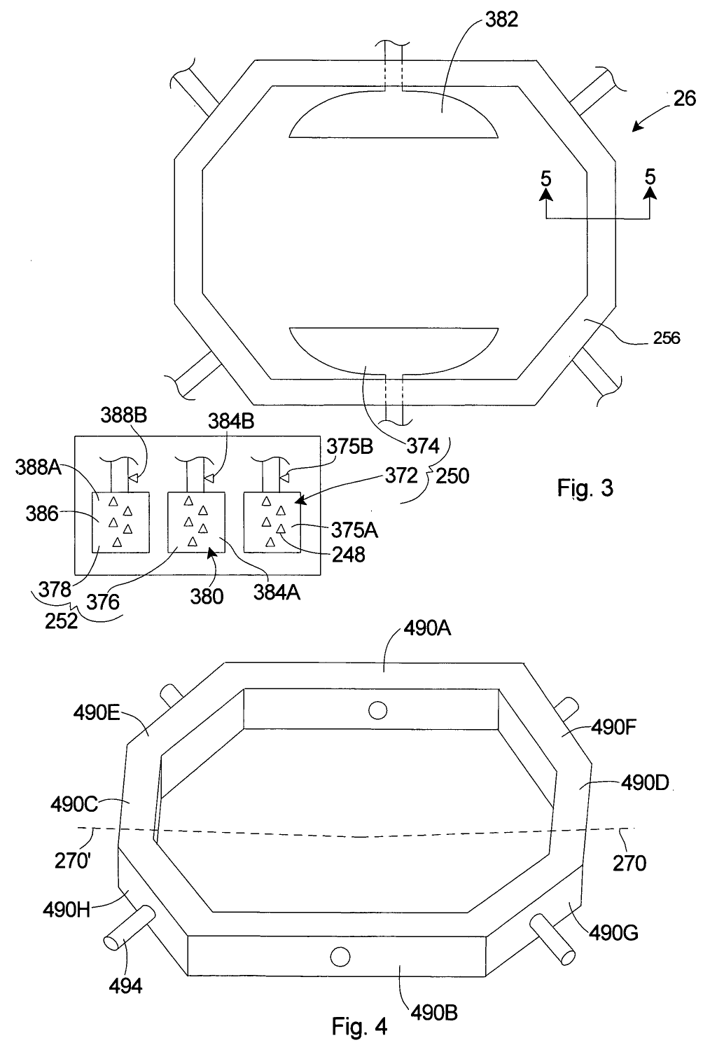

FIG. 3 is a top plan illustration of the fluid supply system 26 including the fluid delivery system 250 and the fluid recovery system 252. In one embodiment, it is desired to completely fill the exposure area with the immersion fluid 248. In fact, to make sure that this area remains filled with the immersion fluid 248 and not some other fluid, the area is overfilled. In other words, the immersion fluid 248 is continuously pumped into the gap 246 (illustrated in FIG. 2B) with the fluid delivery system 250 at a first rate and is deliberately pumped out with the fluid recovery system 252 at a second rate that is less than the first rate. This keeps the gap 246 (illustrated in FIG. 2B) filled with pure immersion fluid 248. In alternative embodiments, the first rate is at least approximately 10, 20, 30, 40 or 50 percent greater than the second rate.

In FIG. 3, the fluid delivery system 250 includes a fluid source 372 and a fluid outlet 374 that is in fluid communication with the fluid source 372. The fluid source 372 delivers pressurized immersion fluid 248 to the fluid outlet 374. The fluid source 372 can include one or more fluid reservoirs 375A that retain the immersion fluid 248 and one or more fluid pumps 375B. The fluid outlet 374 is positioned within the fluid barrier 256 and can include one or more nozzles, or another distribution system such as a channel. Multiple fluid outlets 374 may be placed on both sides or several points at or near the gap 246.

The type of immersion fluid 248 can be varied to suit the design requirements of the apparatus. In one embodiment, the immersion fluid 248 is Nitrogen. Alternatively, for example, the immersion fluid 248 can be Argon, Helium, water, or another type of fluid.

The fluid recovery system 252 includes a first recovery system 376 and a second recovery system 378 that cooperate to capture the immersion fluid 248 released into the gap 246. In one embodiment, the first recovery system 376 includes a first low pressure source 380 and a fluid inlet 382.

The first low pressure source 380 draws the immersion fluid 248 via the fluid inlet 382 from the gap 246. The first low pressure source 380 can include one or more fluid reservoirs 384A that retain the recovered immersion fluid 248 and one or more vacuum pumps 384B. The fluid inlet 382 is positioned within the fluid barrier 256 and can include one or more apertures or channels. Multiple fluid inlets 382 may be placed at several points at or near the gap 246.

The second recovery system 378 includes a second low pressure source 386 and the fluid barrier 256. The second low pressure source 386 can include one or more fluid reservoirs 388A that retain the recovered immersion fluid 248 and one or more vacuum pumps 388B.

The design of the barrier 256 can vary according to the design of the rest of the components of the apparatus 10. In one embodiment, the barrier 256 restricts the flow of the immersion fluid 248 from the gap 246 and allows for the recovery of the immersion fluid 248 that escapes into the movement gap 258 (illustrated in FIG. 2A) between the wafer 30 (illustrated in FIG. 2A) and the barrier 256.

In one embodiment, the fluid barrier 256 encircles and runs entirely around the exposure area 246. Alternatively, for example, the fluid barrier 256 can be positioned around only a portion of the exposure area 246.

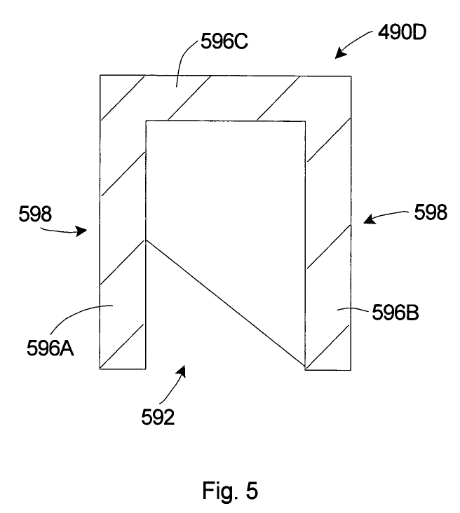

FIG. 4 illustrates a perspective view of the barrier 256 and FIG. 5 is a cross-sectional view taken from FIG. 3. FIG. 4 also illustrates the path of the beam 270 and the reflected beam 270′ through the fluid barrier 256. In this embodiment, the fluid barrier 256 is somewhat octagon frame shaped and includes eight relatively straight regions, namely (i) a top region 490A, (ii) a bottom region 490B, (iii) a left region 490C, (iv) a right region 490D, (v) a top/left region 490E that connects the top region 490A to the left region 490C, (vi) a top/right region 490F that connects the top region 490A to the right region 490D, (vii) a bottom/right region 490G that connects the right region 490D to the bottom region 490B, and (viii) a bottom/left region 490H that connects the left region 490C to the bottom region 490B. It should be noted that the terms top, bottom, left, and right are used merely for convenience and the orientation of the barrier 256 can be rotated.

It should also be noted that the octagon shape is also not necessary and that other shapes can be utilized. Additionally, the left region 490C and the right region 490D do not have to be parallel. Parallel regions may be the preferred embodiment however as they would not affect the calibration of the measurement system 22 (illustrated in FIG. 2A).

In the embodiment illustrated in FIGS. 4 and 5, the fluid barrier 256 also includes a barrier fluid inlet 592, and one or more fluid connectors 494 that connect the barrier fluid inlet 592 in fluid communication with the second low pressure source 386.

Referring to FIG. 5, in this embodiment, the right region 490D includes an inner wall 596A, an outer wall 596B, and a top wall 596C that connects the inner wall 596A to the outer wall 596B. Additionally, the walls 596A-596C cooperate to define a portion of the barrier fluid inlet 592 that is positioned adjacent to the wafer 30 (illustrated in FIG. 2A). In this embodiment, the right region 490D has cross-sectional shape that is somewhat like an upside “U” shape. The other regions 490A-490C, 490E-490H can have a similar shape and design as the right region 490D. With this design, the evacuation barrier fluid inlet 592 extends completely around the exposure area 246.

In one embodiment, one or more of the regions 490A-490H or portions of one or more of the regions 490A-490H includes a transparent area 598. Stated another way, in one embodiment, a portion and/or all of the barrier 256 is made out of an optically transparent material that is substantially transparent so the light 270, 270′ from the measurement system 22 (illustrated in FIG. 2A) can shine through the barrier 256. In this manner traditional optical sensors may still be used in fluid immersion optical systems.

In one embodiment, the left region 490C and the right region 490D each include a transparent area 598 to allow the light 270, 270′ to pass there through. In this embodiment, for example, each wall 596A, 596B or portion of each wall 596A, 596B of each region 490C, 490D includes a transparent area 598. Stated another way, in this embodiment, the barrier 256 includes a transparent area 598 on each side.

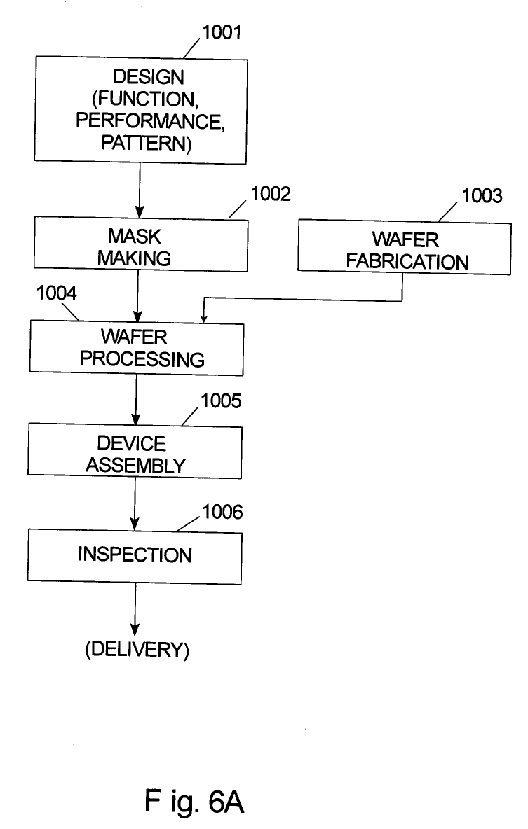

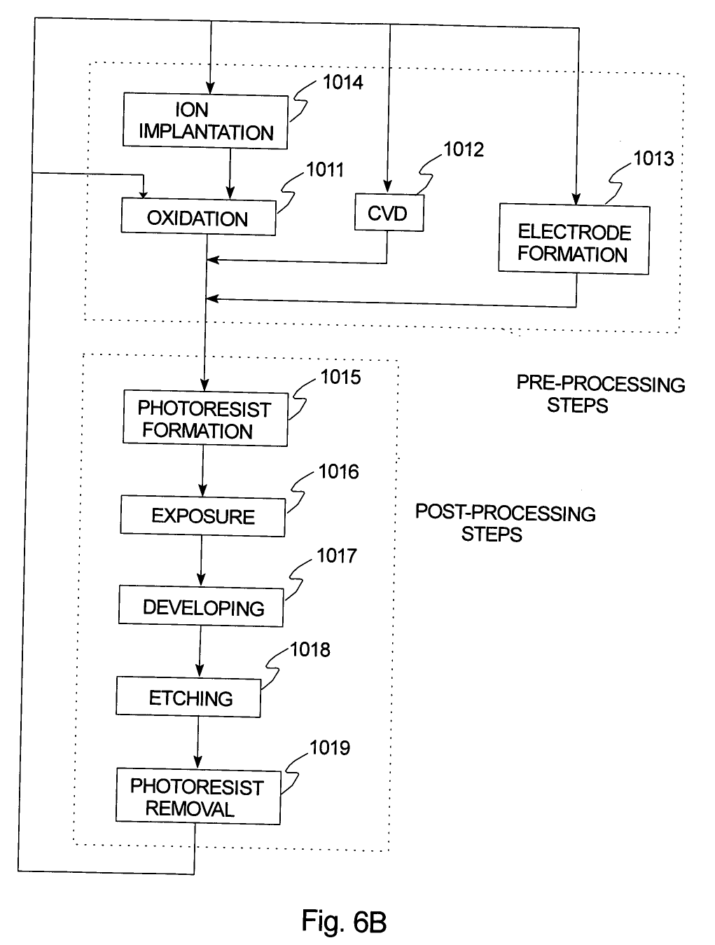

Semiconductor devices can be fabricated using the above described systems, by the process shown generally in FIG. 6A. In step 601 the device's function and performance characteristics are designed. Next, in step 602, a mask (reticle) having a pattern is designed according to the previous designing step, and in a parallel step 603 a wafer is made from a silicon material. The mask pattern designed in step 602 is exposed onto the wafer from step 603 in step 604 by a photolithography system described hereinabove in accordance with the present invention. In step 605 the semiconductor device is assembled (including the dicing process, bonding process and packaging process), finally, the device is then inspected in step 606.

FIG. 6B illustrates a detailed flowchart example of the above-mentioned step 604 in the case of fabricating semiconductor devices. In FIG. 6B, in step 611 (oxidation step), the wafer surface is oxidized. In step 612 (CVD step), an insulation film is formed on the wafer surface. In step 613 (electrode formation step), electrodes are formed on the wafer by vapor deposition. In step 614 (ion implantation step), ions are implanted in the wafer. The above mentioned steps 611-614 form the preprocessing steps for wafers during wafer processing, and selection is made at each step according to processing requirements.

At each stage of wafer processing, when the above-mentioned preprocessing steps have been completed, the following post-processing steps are implemented. During post-processing, first, in step 615 (photoresist formation step), photoresist is applied to a wafer. Next, in step 616 (exposure step), the above-mentioned exposure device is used to transfer the circuit pattern of a mask (reticle) to a wafer. Then in step 617 (developing step), the exposed wafer is developed, and in step 618 (etching step), parts other than residual photoresist (exposed material surface) are removed by etching. In step 619 (photoresist removal step), unnecessary photoresist remaining after etching is removed.

Multiple circuit patterns are formed by repetition of these preprocessing and post-processing steps.

While the particular exposure apparatus 10 as shown and disclosed herein is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A fluid immersion system for controlling an environment in a gap between an optical assembly and a device, the fluid immersion system comprising:
a fluid source that directs an immersion fluid into the gap; and
a fluid barrier that is positioned near the gap, the fluid barrier including a transparent area that is substantially transparent.

2. The fluid immersion system of claim 1 wherein the transparent area has an index of refraction that is approximately equal to an index of refraction of the immersion fluid.

3. The fluid immersion system of claim 1 wherein the transparent area has an index of refraction that is within approximately 0.5 of an index of refraction of the immersion fluid.

4. The fluid immersion system of claim 1 wherein the transparent area has an index of refraction that is within approximately 0.1 of an index of refraction of the immersion fluid.

5. The fluid immersion system of claim 1 wherein the transparent area has a coefficient of extinction of less than approximately 0.06.

6. The fluid immersion system of claim 1 wherein the transparent area has a coefficient of extinction of less than approximately 0.1.

7. The fluid immersion system of claim 1 wherein the transparent area has an index of refraction that is within approximately 5 percent of an index of refraction of the immersion fluid.

8. The fluid immersion system of claim 1 wherein the transparent area has an index of refraction that is within approximately 1 percent of an index of refraction of the immersion fluid.

9. An exposure apparatus for transferring an image to a device, the exposure apparatus comprising: an optical assembly, and the fluid immersion system of claim 1, wherein the barrier encircles a gap between the optical assembly and the device.

10. The exposure apparatus of claim 9 wherein the barrier includes a barrier fluid inlet positioned near the device.

11. The exposure apparatus of claim 10 further comprising a low pressure source that is in fluid communication with the barrier fluid inlet to draw the immersion fluid from the barrier.

12. The exposure apparatus of claim 9 further comprising a measurement system that directs a light beam through the transparent area.

13. A process for manufacturing a device that includes the steps of providing a substrate and transferring an image to the substrate with the exposure apparatus of claim 9.

14. A process for manufacturing a wafer that includes the steps of providing a substrate and transferring an image to the substrate with the exposure apparatus of claim 9.

15. An exposure apparatus for transferring an image to a device, the exposure apparatus comprising:
an optical assembly;
a fluid immersion system for controlling an environment in a gap between the optical assembly and the device, the fluid immersion system including a fluid source that directs an immersion fluid into the gap; and a fluid barrier that is positioned near the gap, the fluid barrier including a transparent area that is substantially transparent; and
a measurement system that directs a light beam through the transparent area.

16. The exposure apparatus of claim 15 wherein the transparent area has an index of refraction that is approximately equal to an index of refraction of the immersion fluid.

17. The exposure apparatus of claim 15 wherein the transparent area has an index of refraction that is within approximately 0.1 of an index of refraction of the immersion fluid.

18. The exposure apparatus of claim 15 wherein the transparent area has a coefficient of extinction of less than approximately 0.1.

19. The exposure apparatus of claim 15 wherein the transparent area has an index of refraction that is within approximately 1 percent of an index of refraction of the immersion fluid.

20. The exposure apparatus of claim 15 wherein the barrier encircles the gap.

21. The exposure apparatus of claim 15 wherein the barrier includes a barrier fluid inlet positioned near the device, and the fluid immersion system further includes a low pressure source that is in fluid communication with the barrier fluid inlet.

22. A process for manufacturing a device that includes the steps of providing a substrate and transferring an image to the substrate with the exposure apparatus of claim 15.

23. A process for manufacturing a wafer that includes the steps of providing a substrate and transferring an image to the substrate with the exposure apparatus of claim 15.

24. A method for making a fluid immersion system for controlling an environment in a gap between an optical assembly and a device, the method comprising the steps of:
directing an immersion fluid into the gap with a fluid source; and
positioning a fluid barrier near the gap, the fluid barrier including a transparent area that is substantially transparent.

25. The method of claim 24 wherein the transparent area has an index of refraction that is approximately equal to an index of refraction of the immersion fluid.

26. The method of claim 24 wherein the transparent area has an index of refraction that is within approximately 0.1 of an index of refraction of the immersion fluid.

27. The method of claim 24 wherein the transparent area has a coefficient of extinction of less than approximately 0.1.

28. The method of claim 24 wherein the transparent area has a coefficient of extinction of less than approximately 0.06.

29. The method of claim 24 wherein the transparent area has an index of refraction that is within approximately 1 percent of an index of refraction of the immersion fluid.

30. A method for making an exposure apparatus for transferring an image to a device, the method comprising the steps of providing an optical assembly, and controlling the environment in a gap between the optical assembly and the device with a fluid immersion system made by the method of claim 24.

31. A process for manufacturing a device that includes the steps of providing a substrate and transferring an image to the substrate with the exposure apparatus made by the method of claim 30.

32. A process for manufacturing a wafer that includes the steps of providing a substrate and transferring an image to the substrate with the exposure apparatus made by the method of claim 30.
